(19) United States
(12) Patent Application Publication
(10) Pub. No.: US 2018/0368343 A1
(43) Pub. Date: Dec. 27, 2018

(54) Sustainable Growing System and Method

(71) Applicant: Greg O'Rourke, San Francisco, CA (US)

(72) Inventor: Greg O'Rourke, San Francisco, CA (US)

(21) Appl. No.: 15/630,726

(22) Filed: Jun. 22, 2017

(51) Int. Cl. A01G 31/02; A01G 33/00; A01G 31/00; C10L 1/02; C10L 1/04; C12M 1/12

(52) U.S. Cl. CPC A01G 31/02; A01G 33/00; A01G 31/001; C12M 31/10; C10L 1/04; C12M 23/04; A01G 31/00; C10L 1/02

(57) ABSTRACT

In one embodiment, a sustainable hydroponic growing system comprises at least one hydroponic growing unit, an algae growing unit configured to produce an algae biomass, a biofuel system configured to process the algae biomass to produce a bioethanol fuel and a solid oxide fuel cell configured to use the bioethanol fuel as at least one source of fuel to produce electrical power for use by the at least one hydroponic growing unit. In one embodiment, the solid oxide fuel cell is further configured to produce steam that serves as a water source for the at least one hydroponic growing unit and/or the algae growing unit.

### FIELD OF THE INVENTION

The invention relates generally to non-soil growing systems and more particularly to a sustainable growing system and method.

### BACKGROUND

Hydroponic growing is a type of indoor agriculture in which plants are grown in a water-based, nutrient-rich solution rather than in soil. In a typical hydroponic growing system, a plant's roots are supported by an inert medium such as perlite and a nutrient-rich solution is circulated to the roots by pumps. Benefits of hydroponic production of crops include total control of the climate (temperature, humidity, light/dark cycles), no need for arable land, faster crop growth, and little to no need for pesticides and herbicides. Hydroponic growing systems also typically require less water and space than traditional agriculture. But hydroponic growing systems require significant amounts of electricity to power lights, pumps, and climate control systems to create the ideal growing environment for the particular crops under production. The nutrients required for plant growth also must be provided in the absence of soil.

The significant amount of electric power required by typical hydroponic growing systems can be a large expense and lead to reduced profits on crops produced. Some efforts to reduce reliance on traditional power, such as using solar power generated on-site, may reduce this expense. But other schemes to reduce power costs may not be appropriate for hydroponic agriculture. For example, would likely not be appropriate for a typical hydroponic growing system to participate in a demand response system of an electric power utility because reducing energy usage during periods of high demand could disrupt climate control systems that maintain optimal temperature and humidity conditions for crop production. Thus there is a need for a more energy efficient hydroponic growing system.

### SUMMARY

In one embodiment, a sustainable hydroponic growing system comprises at least one hydroponic growing unit, an algae growing unit configured to produce an algae biomass, a biofuel system configured to process the algae biomass to produce a bioethanol fuel and a solid oxide fuel cell configured to use the bioethanol fuel as at least one source of fuel to produce electrical power for use by the at least one hydroponic growing unit. In one embodiment, the solid oxide fuel cell is further configured to produce steam that serves as a water source for the at least one hydroponic growing unit and/or the algae growing unit.

In one embodiment, a sustainable growing method comprises operating a solid oxide fuel cell to produce electrical power and water, providing a portion of the electrical power to a hydroponic growing unit and providing a portion of the water to a biofuel reactor, growing an algae biomass and providing the algae biomass to the biofuel reactor, processing the algae biomass by the biofuel reactor to produce bioethanol, and reforming the bioethanol to produce hydrogen as fuel for the solid oxide fuel cell. In one embodiment, the method further comprises providing a portion of the water to the hydroponic growing unit and/or an algae growing unit for growing the algae biomass.

In one embodiment, a sustainable growing system comprises at least one crop growing unit, an algae growing unit configured to produce an algae biomass, a bioreactor configured to process the algae biomass to produce a bioethanol fuel, and a solid oxide fuel cell system configured to process at least the bioethanol fuel to produce electrical power for use by the at least one crop growing unit. In one embodiment, the solid oxide fuel cell system is configured to output water to the at least one crop growing unit, to the algae growing unit, and/or the bioreactor. In one embodiment, the solid oxide fuel cell system comprises a reformer configured to reform at least the bioethanol fuel to produce hydrogen gas and a solid oxide fuel cell configured to process the hydrogen gas to produce electrical power.

### BRIEF DESCRIPTION OF THE DRAWINGS

FIG. 1 is a block diagram of one embodiment of a sustainable growing system, according to the invention.

FIG. 2 is a block diagram of one embodiment of the fuel cell system of the sustainable growing system of FIG. 1, according to the invention.

FIG. 3 is a block diagram of one embodiment of the biofuel system of the sustainable growing system of FIG. 1, according to the invention.

FIG. 4 is a block diagram of one embodiment of the crop and algae system of the sustainable growing system of FIG. 1, according to the invention.

FIG. 5 is a flowchart of method steps for a sustainably growing crops, according to one embodiment of the invention.

### DETAILED DESCRIPTION

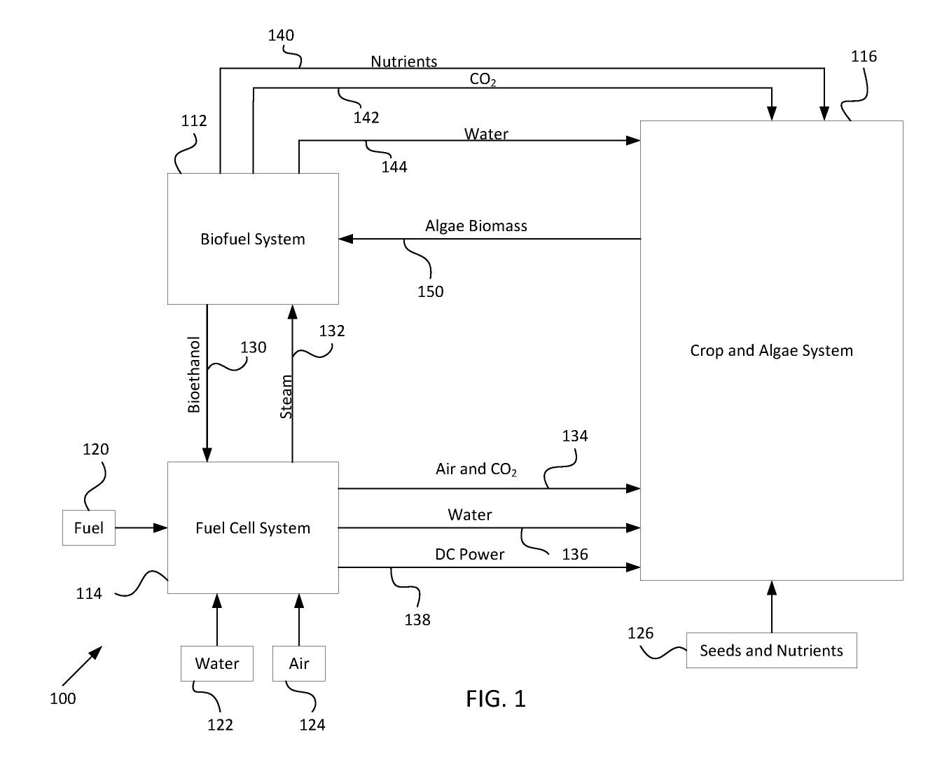

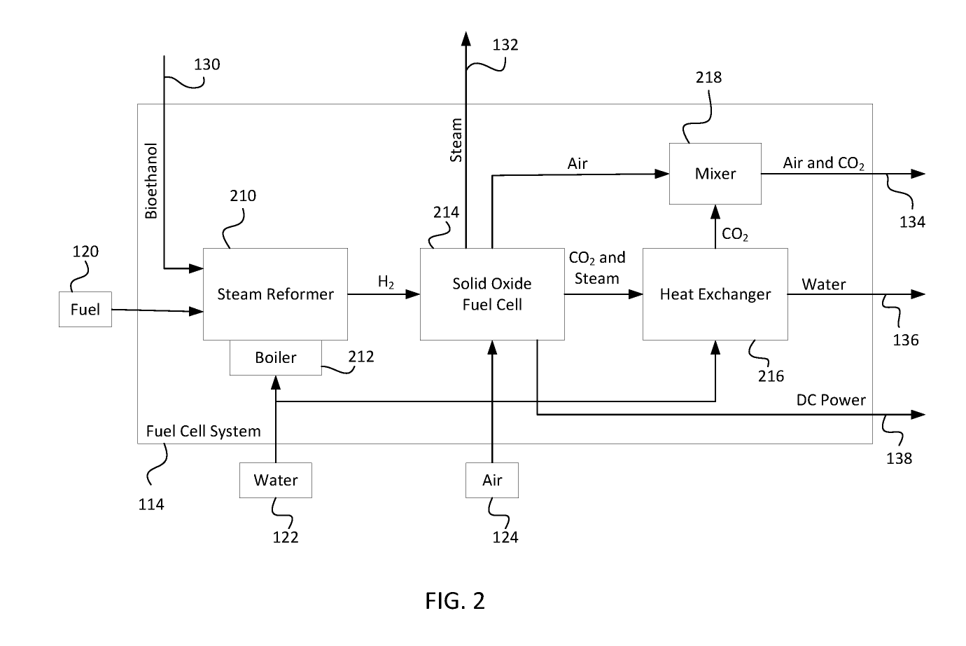

FIG. 1 is a block diagram of one embodiment of a sustainable growing system 100, according to the invention. Sustainable growing system 100 includes, but is not limited to, a biofuel system 112, a fuel cell system 114, and a crop and algae system 116. Fuel cell system 114 receives a fuel such as natural gas or methane from a fuel source 120, water from a water source 122, and air from an air source 124. Fuel cell system 114 outputs heated air and carbon dioxide through a connection 134, heated water and/or steam through a connection 136, and direct current (DC) power through a connection 138 to crop and algae system 116. Fuel cell system 114 also outputs steam through a connection 132 to biofuel system 112. Fuel cell system 114 is described further below in conjunction with FIG. 2.

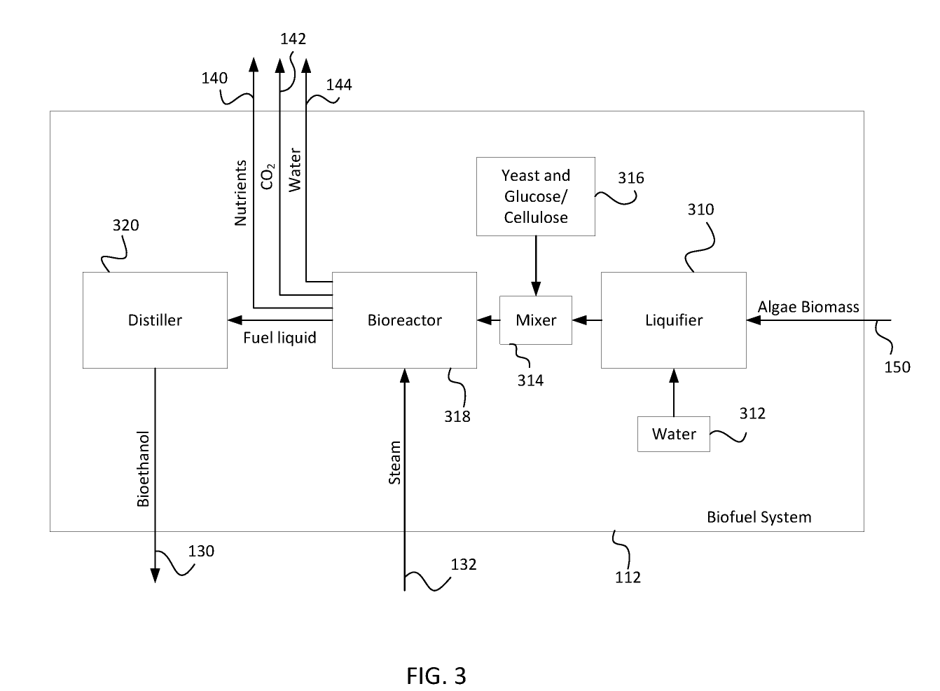

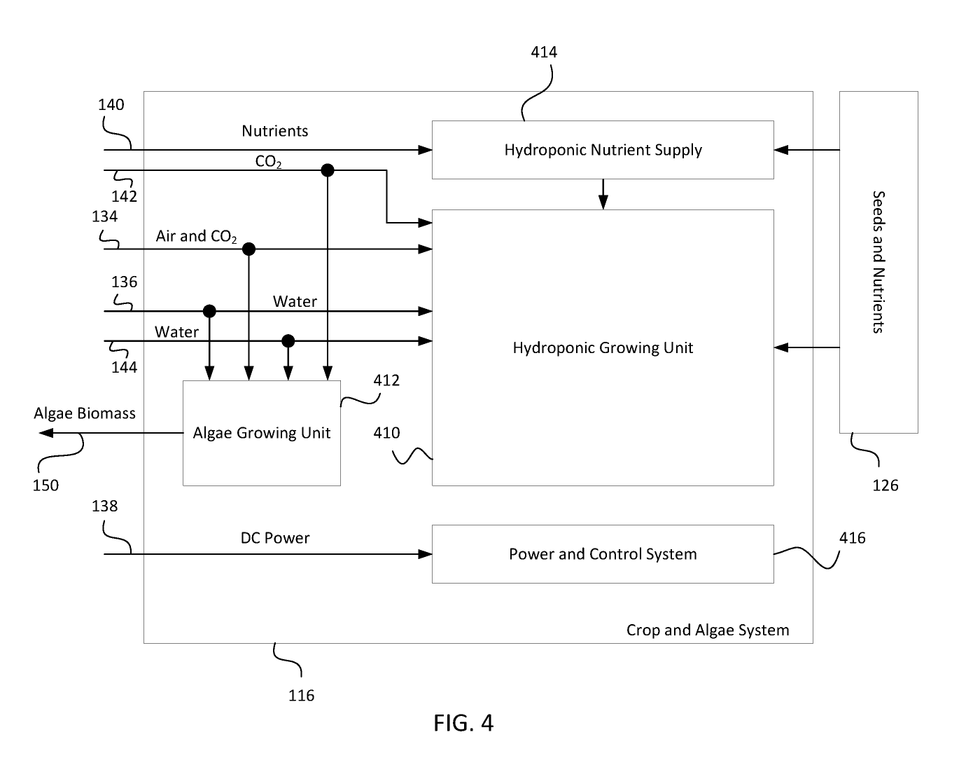

Biofuel system 112 receives an algae biomass through a connection 150 from crop and algae system 116. Biofuel system 112 processes the algae biomass using steam from fuel cell system 114 to produce bioethanol that is output through a connection 130 to fuel cell system 114. In one embodiment, the bioethanol produced by biofuel system 112 supplements fuel from fuel source 120. In another embodiment, bioethanol produced by biofuel system 112 is the sole source of fuel for fuel cell system 114. Biofuel system 112 also produces nutrients, water, and carbon dioxide that are output through connections 140, 142, 144 to crop and algae system 116. Biofuel system 112 is described further below in conjunction with FIG. 3. Crop and algae system 116 receives seeds and nutrients from a seeds and nutrients source 126. Crop and algae system 116 produces the algae biomass input to biofuel system 112 and produces one or more crops (not shown) such as fruits, vegetables, or herbs. Crop and algae system 116 is further described below in conjunction with FIG. 4.

As shown in FIG. 1, in addition to providing electrical energy to crop and algae system 116, fuel cell system 114 provides the byproducts of its energy-conversion processes including water and heat to crop and algae system 116 and biofuel system 112. Sustainable growing system 100 uses what are typically considered to be the waste products of fuel cell system 114 as resources for crop and algae system 116 and biofuel system 112. Similarly, sustainable growing system 100 uses the byproducts of biofuel system 112, including nutrients such as biochar and bio-oil, water, and carbon dioxide, as resources for crop and algae system 116. Biofuel system 112 provides a supply of bioethanol as a sustainable source of fuel to power at least in part the production of electricity by fuel cell system 114. In one embodiment, the bio-oil byproduct of biofuel system 112 is further refined to provide a biofuel, such as biodiesel, for use by fuel cell system 114.

FIG. 2 is a block diagram of one embodiment of the fuel cell system 114 of the sustainable growing system 100 of FIG. 1, according to the invention. Fuel cell system 114 includes, but is not limited to, a steam reformer 210, a boiler 212, a solid oxide fuel cell 214, a heat exchanger 216, and a mixer 218. Boiler 212 heats water from water source 122 to produce steam that is input to steam reformer 210. Steam reformer 210 steam reforms fuel such as natural gas containing methane from fuel source 120 and/or bioethanol from biofuel system 112 to produce hydrogen gas. In one embodiment, the bioethanol produced by biofuel system 112 supplements fuel from fuel source 120. In another embodiment, bioethanol produced by biofuel system 112 is the sole source of fuel input to steam reformer 210. Solid oxide fuel cell 214 receives the hydrogen gas from steam reformer 210 and receives oxygen-containing air from air source 124. Solid oxide fuel cell 214 converts the hydrogen gas to electrical energy and heat by electrochemically combining the hydrogen gas fuel with the oxygen-containing air. In solid oxide fuel cell 214, oxide ions combine with hydrogen to form steam and carbon dioxide, freeing electrons that provide DC power output to connection 138. Solid oxide fuel cell 214 outputs steam to biofuel system 112 through connection 132, and outputs steam and carbon dioxide to heat exchanger 216. Solid oxide fuel cell 214 also outputs heated air to mixer 218. In one embodiment, solid oxide fuel cell 214 is a tubular fuel cell with a power rating of about 500 W; however other types of solid oxide fuel cells such as a planar fuel cell and other types of fuel cells such as proton exchange membrane (PEM) cells are within the scope of the invention.

Heat exchanger 216 receives water from water source 122 and heats the water, preferably to a temperature in the range of about 30-35° C., with steam from solid oxide fuel cell 214. Heat exchanger 216 outputs the heated water to crop and algae system 116 through connection 136. Heat exchanger 216 also outputs carbon dioxide to mixer 218, which mixes the carbon dioxide with air from solid oxide fuel cell 214. Mixer 218 outputs a mixture of air and carbon dioxide to crop and algae system 116 through connection 134. In one embodiment, fuel cell system 114 does not include heat exchanger 216 or mixer 218, and the air, carbon dioxide, and steam produced by solid oxide fuel cell 214 is output directly to crop and algae system 116.

FIG. 3 is a block diagram of one embodiment of the biofuel system 112 of the sustainable growing system 100 of FIG. 1, according to the invention. Biofuel system 112 includes, but is not limited to, a liquefier 310, a mixer 314, a bioreactor 318, and a distiller 320. Algae biomass from crop and algae system 116 is input to liquefier 310, which uses water from a water source 312 to liquefy the algae biomass. The liquefied algae biomass is input to mixer 314, which mixes the liquefied algae biomass with yeast and glucose and/or cellulose from a yeast and glucose/cellulose source 316. Mixer 314 outputs the algae-yeast-glucose/cellulose mixture to bioreactor 318. Bioreactor 318 then breaks down the algae-yeast-glucose/cellulose mixture to release its carbohydrates. Bioreactor 318 ferments the broken-down algae-yeast-glucose/cellulose mixture to produce a fuel liquid that is output to distiller 320. The fermentation process converts the carbohydrates (sugars) in the algae and glucose/cellulose into ethyl alcohol (ethanol). Bioreactor 218 operates at a high temperature, which causes internal pressurization to accelerate the reactor processes. Distiller 320 separates the bioethanol from the fuel liquid using a distillation process and outputs the bioethanol to fuel cell system 114.

Byproducts from the fermentation process of bioreactor 318 are output to crop and algae system 116. Bioreactor 318 separates the byproducts including nutrients, carbon dioxide, and water from the fuel liquid that is output to distiller 320. Nutrients such as biochar (black carbon) are output to crop and algae system through connection 140. Bioreactor 318 outputs carbon dioxide through connection 142 and water through connection 144 to crop and algae system 116.

FIG. 4 is a block diagram of one embodiment of the crop and algae system 116 of the sustainable growing system 100 of FIG. 1, according to the invention. Crop and algae system 116 includes, but is not limited to, at least one hydroponic growing unit 410, an algae growing unit 412, a hydroponic nutrient supply 414, and a power and control system 416. Hydroponic growing unit 410 produces one or more crops such as vegetables, fruits, or herbs. In one embodiment, crop and algae system 116 includes a plurality of hydroponic growing units 410. Hydroponic growing unit 410 is configured to grow plants in a water-based, nutrient-rich solution where the plants' root systems are not supported by soil. Any type of hydroponic system such as a deep water culture system, a nutrient film technique system, an aeroponics system, an ebb and flow system, a wicking system, a dripping system, or a combination of these systems is within the scope of the invention. Hydroponic growing unit 410 mixes nutrients from hydroponic nutrient supply 414 and water from fuel cell system 114 and biofuel system 112 to produce a nutrient solution for the crops under production. Hydroponic nutrient supply 414 receives nutrients from seed and nutrient source 126 and from biofuel system 112. Hydroponic growing unit 410 includes systems for lighting, circulation of nutrient solution, climate control, and monitoring of growing conditions.

Algae growing unit 412 can be implemented as any appropriate system for growing algae such as an open pond or a closed-loop system. Algae growing unit 412 receives water, air, and carbon dioxide from fuel cell system 114 and/or biofuel system 112. Growing any strain of algae with a high carbohydrate content is within the scope of the invention. In one embodiment, algae growing unit 412 also includes a press or other mechanism (not shown) for extracting oils from the raw algae to produce a dry algae biomass that is output to biofuel system 112.

Power and control system 416 receives DC power from fuel cell system 114 and provides power and control signals to all electrical systems for algae growing unit 412 and hydroponic growing unit 410, including but not limited to lighting, pumps, and climate systems. For example, power and control system 416 provides power to fans (not shown) that control temperature and circulation of air and carbon dioxide in hydroponic growing unit 410. Power and control system 416 also provides power and control signals to pumps (not shown) that provide the nutrient solution to the crops under production in hydroponic growing unit 410.

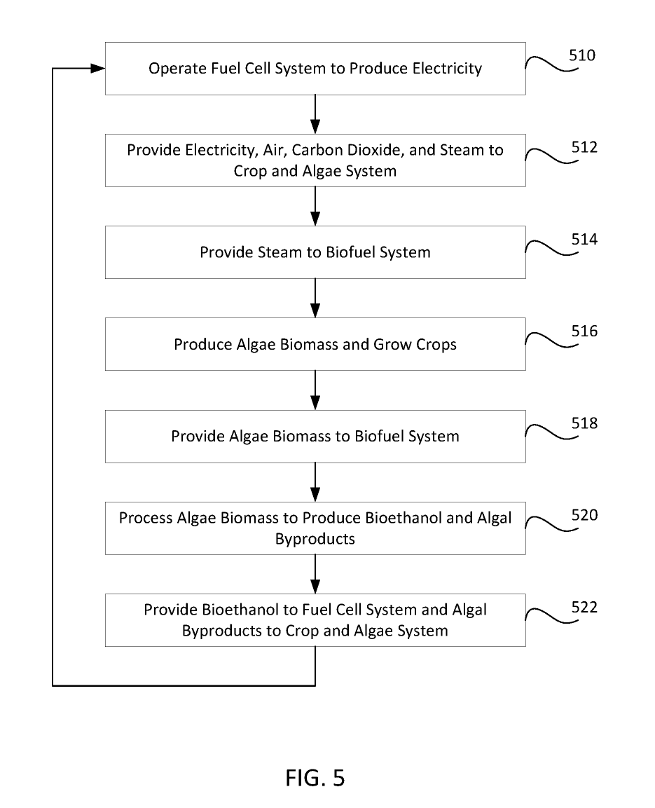

FIG. 5 is a flowchart of method steps for sustainably growing crops, according to one embodiment of the invention. In step 510, a fuel cell system is operated to produce electricity. In step 512, the electricity produced by the fuel cell system is provided to a crop and algae system, and air, carbon dioxide, and steam produced by the fuel cell system as byproducts are also provided to the crop and algae system. In step 514, steam produced by the fuel cell system is provided to a bioreactor of a biofuel system. In step 516, crops are grown and an algae biomass is produced by the crop and algae system. In one embodiment, crop and algae system is a hydroponic growing system that include an algae growing unit. In step 518, the algae biomass produced by the crop and algae system is provided to the biofuel system as raw material for the production of bioethanol. In step 520, the biofuel system processes the algae biomass to produce bioethanol and algal byproducts such as biochar, bio-oil, water, and carbon dioxide. In step 522, the bioethanol is provided to the fuel cell system as a supplement to and/or replacement for other fuels such as methane, and the algal byproducts are provided to the crop and algae system. The method then returns to step 510 where the fuel cell system produces electricity fueled at least in part by the bioethanol produced by the biofuel system.

The invention has been described above with reference to specific embodiments. It will, however, be evident that various modifications and changes may be made thereto without departing from the broader spirit and scope of the invention as set forth in the appended claims. The foregoing description and drawings are, accordingly, to be regarded in an illustrative rather than a restrictive sense.

What is claimed is:

1. A hydroponic growing system comprising:
at least one hydroponic growing unit;
an algae growing unit configured to produce an algae biomass;
a biofuel system configured to process the algae biomass to produce a bioethanol fuel; and
a solid oxide fuel cell configured to use the bioethanol fuel as at least one source of fuel to produce electrical power for use by the at least one hydroponic growing unit.

2. The hydroponic growing system of claim 1, wherein the solid oxide fuel cell is further configured to produce steam that serves as a water source for the at least one hydroponic growing unit.

3. The hydroponic growing system of claim 1, wherein the solid oxide fuel cell is further configured to produce steam that serves as a water source for the algae growing unit.

4. The hydroponic growing system of claim 1, wherein the solid oxide fuel cell is further configured to produce steam that is used by the biofuel system to process the algae biomass.

5. The hydroponic growing system of claim 1, wherein the biofuel system is configured to output byproducts of a bioreactor to the at least one hydroponic growing unit.

6. The hydroponic growing system of claim 5, wherein the byproducts of the bioreactor include one or more of nutrients, water, and carbon dioxide.

7. A method comprising:
operating a solid oxide fuel cell to produce electrical power and water;
providing a portion of the electrical power to a hydroponic growing unit and providing a portion of the water to a biofuel reactor;
growing an algae biomass and providing the algae biomass to the biofuel reactor;
processing the algae biomass by the biofuel reactor to produce bioethanol; and
reforming the bioethanol to produce hydrogen as fuel for the solid oxide fuel cell.

8. The method of claim 7, further comprising providing a portion of the water to the hydroponic growing unit.

9. The method of claim 7, further comprising providing a portion of the water to an algae growing unit for growing the algae biomass.

10. The method of claim 7, further comprising mixing the algae biomass with glucose or cellulose prior to processing by the biofuel reactor.

11. The method of claim 7, further comprising providing a portion of the electrical power to an algae growing unit for growing the algae biomass.

12. The method of claim 7, further comprising providing byproducts of the bioreactor to the hydroponic growing unit.

13. A growing system comprising:
at least one crop growing unit;
an algae growing unit configured to produce an algae biomass;
a bioreactor configured to process the algae biomass to produce a bioethanol fuel; and
a solid oxide fuel cell system configured to process at least the bioethanol fuel to produce electrical power for use by the at least one crop growing unit.

14. The growing system of claim 13, wherein the solid oxide fuel cell system is further configured to produce electrical power for use by the algae growing unit.

15. The growing system of claim 13, wherein the solid oxide fuel cell system is configured to output water to the at least one crop growing unit.

16. The growing system of claim 13, wherein the solid oxide fuel cell system is configured to output water to the algae growing unit.

17. The growing system of claim 13, wherein the solid oxide fuel cell system is configured to output water to the bioreactor.

18. The growing system of claim 13, wherein the bioreactor is further configured to provide byproducts of a fermentation process to the at least one crop growing unit.

19. The growing system of claim 18, wherein the byproducts of the fermentation process include one or more of nutrients, water, and carbon dioxide.

20. The growing system of claim 13, wherein the solid oxide fuel cell system comprises a reformer configured to reform at least the bioethanol fuel to produce hydrogen gas and a solid oxide fuel cell configured to process the hydrogen gas to produce electrical power.
